(19) United States
(12) Patent Application Publication
(10) Pub. No.: US 2008/0287891 A1
(43) Pub. Date: Nov. 20, 2008

(54) Device for irrigating and inspecting a wound

(76) Inventor: Kevin L. Foster, Pasadena, CA (US)

Correspondence Address: Frederick Gotha, Suite 800, 301 East Colorado Blvd., Pasadena, CA, 91101, US

(21) Appl. No.: 12/151,346

(22) Filed: May 6, 2008

Related U.S. Application Data
(63) Continuation-in-part of application No. 11/804,116, filed on May 17, 2007.

(51) Int. Cl. A61M 35/00; A61B 5/00

(52) U.S. Cl. 604/310; 600/300

(57) ABSTRACT

The invention relates to a device for more effectively performing wound irrigation and cleansing. The device consists of a coupling member that may be releasably coupled by threaded engagement with the dispensing outlet of a manually compressible container having a reservoir of cleansing fluid. The coupling member has an upstream end portion containing a void region which is in fluid communication with the reservoir after coupling. A magnifying lens is carried by the coupling member and is located intermediate the upstream and downstream ends of the coupling member. The magnifying lens has a multiplicity of orifices adjacent its peripheral edge where the orifices communicate with both the void region and a chamber region. The chamber region is located downstream of the lens and has an opening at its downstream end to permit cleansing fluid to be delivered to the wound upon manually compressing the container. After cleansing the wound, the coupling member is de-coupled from the container and the wound inspected through the magnifying lens for contaminants or debris.

This is a continuation-in-part application of application Ser. No. 11/804,116 filed May 17, 2007.

### FIELD OF THE INVENTION

This invention relates to a device for more effectively performing wound irrigation and cleansing of contaminants by utilizing a magnification lens.

### BACKGROUND OF THE INVENTION

Open wounds or lacerations generally contain contaminants and debris that unless timely removed will cause the wound to become infected. The presence of residual contaminants or debris may be the source of bacterial build-up and consequent infection even after the wound has been irrigated with a cleansing solution. The most common procedure for cleansing wounds is by irrigation methods well known in the prior art such as those described in the U.S. Pat. No. 6,468,253 and U.S. Pat. No. 4,769,003. The discharge nozzles, bulb type syringes, and gravity flow devices of the prior art rely upon a steady volumetric stream of cleansing fluid during the irrigation process to wash the wound clean of debris and contaminants. Each of these procedures requires the wound to be visually examined by the naked eye after each irrigation in order to assure that the contaminants have been substantially removed and the wound cleansed. If no contaminants are seen the wound is considered cleansed and irrigation is discontinued; the presence, however, of contaminants or debris that was undetected by visual inspection may be a source for a rapid bacterial build-up and consequent infection. It is therefore desirable to not only provide an inexpensive device that delivers a sufficient stream of cleansing fluid to wash a wound but to also enable a visual inspection of the wound that detects the presence of minute contaminants and debris which would not be ordinarily identifiable by a naked eye inspection and to determine the physical dimension of the wound.

### SUMMARY OF THE INVENTION

There is therefore, provided according to the present invention, a device for irrigating an open wound or laceration with cleansing fluid and for magnifying the wound to identify residual contaminants and debris which would ordinarily be unidentifiable by a naked eye visual inspection.

The present invention is directed to a device for irrigating an open wound or laceration with cleansing fluid contained in the reservoir of a manually compressible container of the type having a dispensing outlet which is in fluid communication with the reservoir and for magnification of and visually examining the magnified wound to detect the presence of minute contaminants and debris. The device consists of a coupling member having an axis of elongation, an upstream end portion, and downstream end portion, where the upstream portion is so dimensioned and proportioned to permit the coupling member to releasably couple with the dispensing outlet of the container. The upstream end portion of the coupling member has an axially extending void region that is in fluid communication with the reservoir when the coupling member couples with the dispensing outlet of the compressible container.

To permit magnification of the open wound, a magnifying lens is carried by the coupling member intermediate the upstream end portion and the downstream end portion where the upstream surface of the lens forms a downstream boundary of the void region and the downstream surface of the lens forms an upstream boundary of the chamber region located within the downstream end portion of the coupling member. The magnifying lens has a multiplicity of orifices radially spaced adjacent the peripheral edge of said lens to minimize lens distortion and to permit the cleansing fluid to flow from the void region to the chamber region upon sufficient compression of the container.

In the preferred embodiment of this invention, the downstream end portion of the coupling member comprises a splash shield circumferentially enclosing the chamber region where the downstream terminus of the splash shield is so contoured and dimensioned to permit cleansing fluid to drain radially through drain ports which extend radially through downstream terminus. In another embodiment, the downstream terminus of the splash shield has radially and circumferentially spaced indicia adjacent the periphery of the downstream terminus and located between the drain ports for measuring the physical dimension of the wound.

### BRIEF DESCRIPTION OF THE DRAWINGS

These and other features and advantages will become appreciated as the same become better understood with reference to the following specification, claims and drawings wherein:

FIG. 1 is a perspective view of this invention illustrating the coupling member and wound cleansing fluid container in spaced relationship.

FIG. 2 is a perspective view of this invention illustrating the coupling member and cleansing fluid container in coupled relationship.

FIG. 3 is a prospective view of this invention illustrating the coupled cleansing device near the surface area of a wound.

FIG. 4 is a prospective view of this invention illustrating the coupled device in bearing engagement with the surface area of a wound.

FIG. 5 is a perspective view of the coupling member of this invention uncoupled from the cleansing fluid container for magnified examination of the wound to detect residual debris or contaminants remaining after cleansing.

FIG. 6 is a part-front view of the coupling member of this invention detached from the fluid cleansing container.

FIG. 7 is a cross-sectional view of FIG. 6

FIG. 8 is perspective view of the coupling member of this invention illustrating the indicia for measurement of the physical dimensions of a wound.

FIG. 9 is a bottom view of FIG. 8 illustrating rotation of the coupling member to ascertain the physical dimension of the wound.

### DETAILED DESCRIPTION

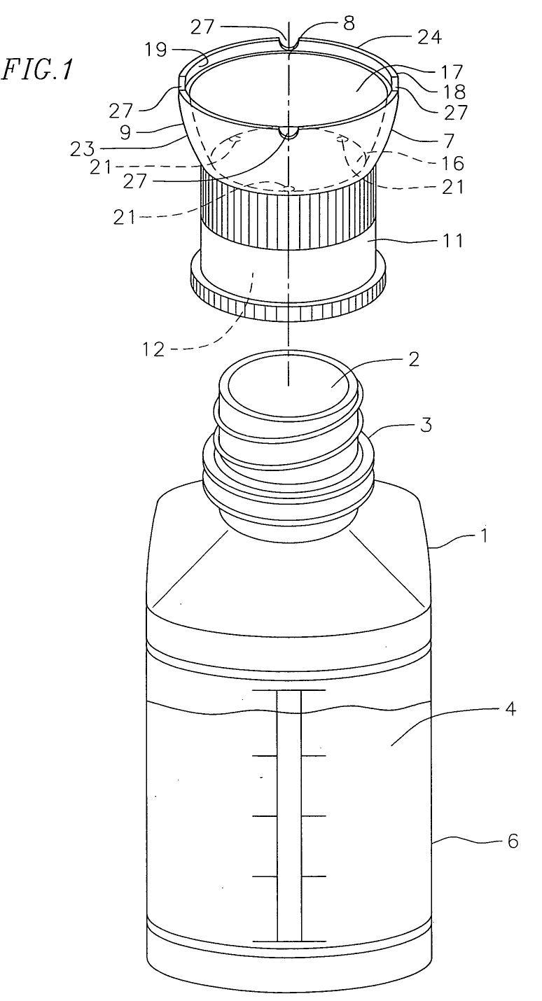

FIG. 1 is a perspective view illustrating the cleansing fluid container 1 having a dispensing outlet 2 and a neck portion 3 where neck portion 3 ordinarily is threaded or otherwise adapted for closure by a cap (not shown) that seals the outlet and is removed when the cleansing fluid 4 is to be dispensed. Container 1 is commonly made of a resilient plastic material and the cleansing fluid 4 is generally a saline type solution. As can further be seen in FIG. 1, container 1 has a reservoir region 6 which retains the cleansing fluid 4. In order to dispense the cleansing fluid from the reservoir, other than by gravity, container 1 is resiliently constructed and manually compressible such that the cleansing fluid is dispensed under pressure from the reservoir through dispensing outlet 2.

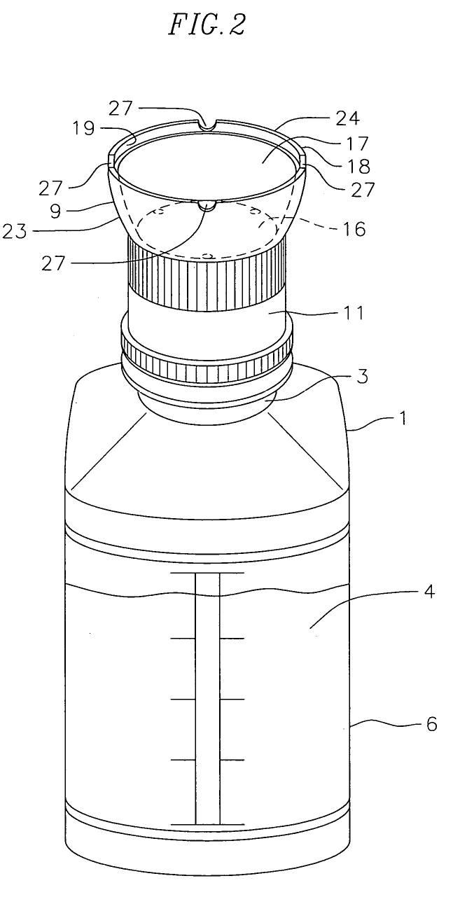

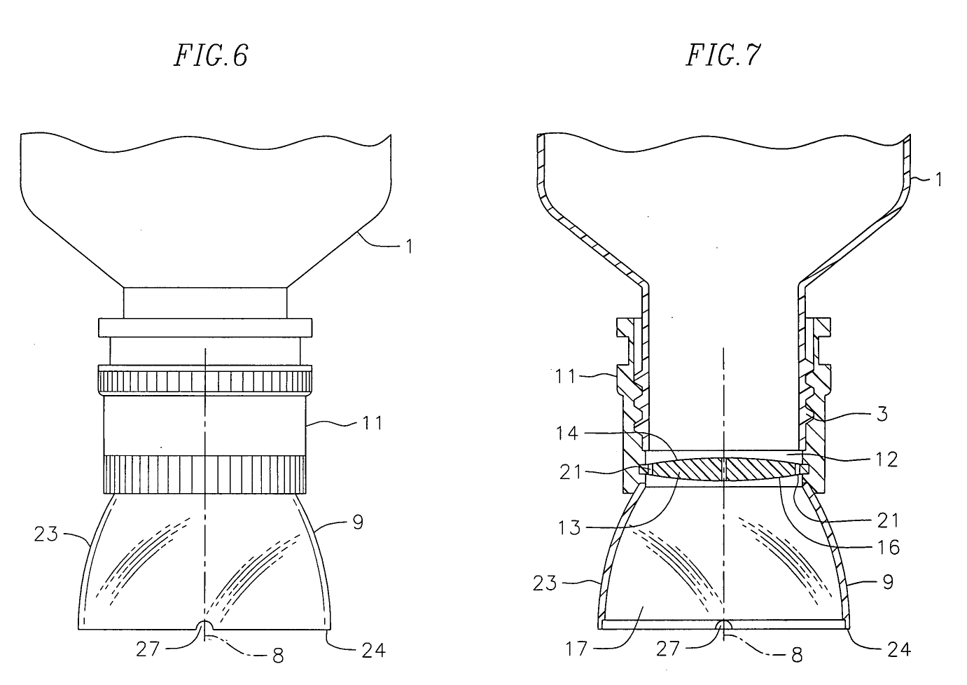

Referring to FIG. 1, coupling member 7 of this invention is shown in perspective and in explosive association axially removed from container 1. Although not shown in FIG. 1, but can be seen in FIG. 7, coupling member 7 may be threaded internally or otherwise adapted to sealingly couple with dispensing outlet 2 of neck portion 3. Coupling member 7 has an axis of elongation 8 and in the preferred embodiment, is radially symmetrical to axis of elongation 8. Coupling member 7 also has a downstream end portion 9 and an upstream end portion end portion 11 where upstream end portion 11 has an axially extending void region 12 therein. By referring to FIG. 7, it can be seen that axially extending void region 12 is bounded by a magnifying lens 13, having an upstream surface 14 and a downstream surface 16 where magnifying lens 13 is integrally carried by coupling member 7 and is located intermediate upstream end portion 11 and downstream end portion 9. As more clearly shown in FIGS. 1, 2 and 7, coupling member 7 has an axially extending chamber region 17 that in the preferred embodiment is radially symmetrical to axis of elongation 8. Downstream surface 16 of lens 13 forms the upstream boundary of chamber region 17. The terminal downstream end 18 of chamber region 17 has an opening 19 to permit cleansing fluid to flow past downstream end 18 when cleansing fluid container 1 is sufficiently compressed to force the fluid through the orifices adjacent the periphery of magnifying lens 13.

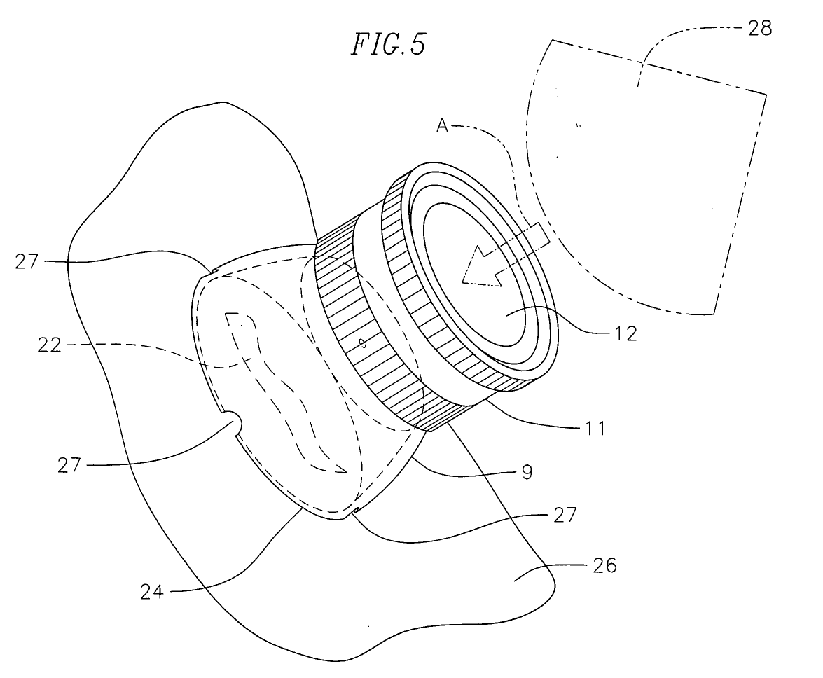

Magnifying lens 13, as can be seen in FIGS. 1 and 7, has a multiplicity of orifices 21 that extend through the lens and have openings in downstream and upstream surfaces 16 and 14 of the lens. Orifices 21 are so dimensioned and proportioned to permit cleansing fluid to flow through under fluid pressure applied by compressing container 1 resulting in a steady stream of cleansing fluid reaching wound 22. In the embodiment of this invention shown in FIGS. 1-7, the downstream end portion 9 is a splash skirt 23 having a terminal edge 24 that is so contoured to permit the edge to flushly engage the area 26 surrounding wound 22. A multiplicity of drain ports 27 located adjacent terminal edge 24 permit cleansing fluid to drain from the wound site through the terminal edge to during the wound cleansing.

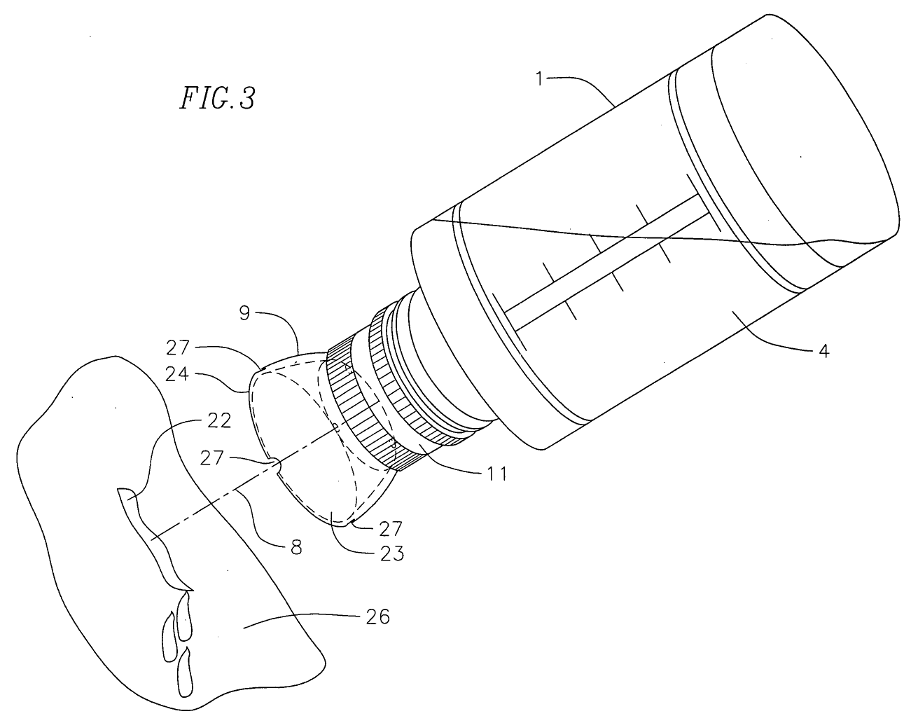

FIG. 3 illustrates an initial step in the sequence of cleansing wound 22. As can be seen, container 1 is inverted with coupling member 7 approaching the wound site in coupled relationship with container 1.

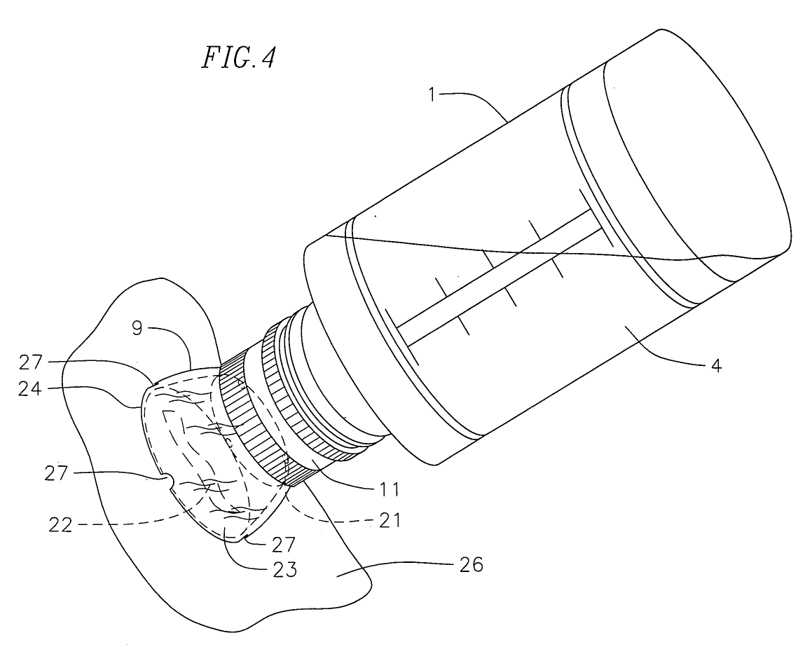

FIG. 4 illustrates another step in the sequence of cleansing the wound where terminal edge 24 is in bearing engagement with wound area 26 and surrounding wound 22. In the preferred embodiment, splash skirt 23 has drain ports 27 to permit drainage of the cleansing fluid while maintaining bearing engagement with wound area 26. In another embodiment, not shown in the drawings, downstream end portion 9 may have a terminal edge that does not contain drain ports.

FIG. 5 illustrates the examination sequence of cleansing the wound. As can be seen in FIG. 5, after container 1 has been decoupled from coupling member 7, terminal edge 24 is placed in bearing engagement with wound area 26 to permit visual inspection of the wound through magnifying lens 13. By observing the wound through void region 12 in the direction of arrow A, the observer 28 will see a magnified image of wound 22 that permits the observer to detect contaminants and debris that would not be readily identifiable through a naked eye inspection of the wound. In the event debris or contaminants are found, coupling member 7 may be re-coupled at the dispensing outlet 2 of neck portion 3 and the sequence repeated.

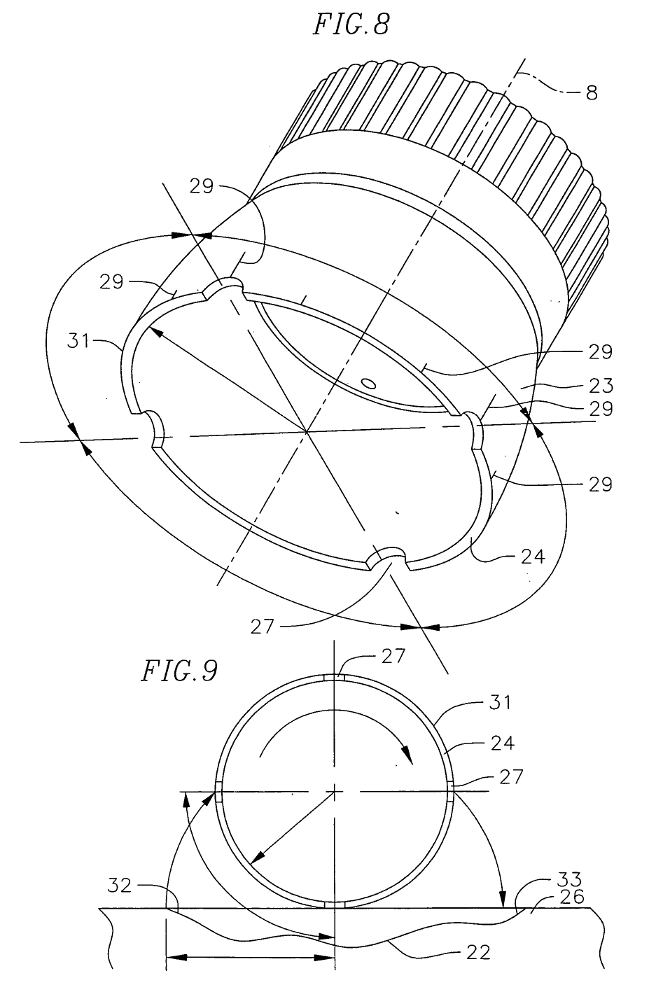

Another embodiment of this invention is shown in FIGS. 8 and 9. In this embodiment, the downstream terminal edge 24 of splash skirt 23 has a multiplicity of indicia 29 which are radially and circumferentially spaced adjacent downstream terminal edge 24. With the outer diameter of terminal edge 24 known, the indicia may be spaced apart a known distance along circumference 31 of terminal edge 24. Thus, the length of wound 22 may be measured by rolling coupling member 3 from a first end 32 of wound 22 to the second end 33 of the wound as illustrated in FIGS. 8 and 9. As can be seen in FIG. 8, drain parts 27 are 90° apart and the circumferential distance between each indicia 29 is 1/16 of the outer circumference 31 of downstream terminal edge 24.

The indicia 29 may therefore be spaced radially a predetermined circumferential distance apart in fixed and integral relationship with splash skirt 23. To measure the physical dimensions of the wound 22, the coupling member 7 is placed adjacent the wound preferably with a drain port 27 over the first end 32 of the wound and then rolled along the length of the wound to the second end 33 of the wound. The indicia may be in fractional value or otherwise numbered to convey to the user the circumferential distance between them.

While I have shown and described embodiments of a device for irrigating and inspecting a wound, it is to be understood that the invention is subject to many modifications without departing from the scope and spirit of the claims recited herein.

What is claimed is:

1. A device for both irrigating a laceration with irrigation fluid contained in the reservoir of a manually compressible container of the type having a neck portion with a dispensing outlet in fluid communication with the container reservoir and for examining the laceration for residual contaminants and debris, comprising:
(a) a coupling member having an axis of elongation, an upstream end portion, and a downstream end portion, where said upstream end portion is so dimensioned and proportioned to permit said upstream end portion to releasably couple with said dispensing outlet, said upstream end portion of said coupling member having a axially extending void region therein where said reservoir is in fluid communication with said axially extending void region upon coupling of said coupling member with said dispensing outlet;
(b) a magnifying lens having a peripheral edge carried by said coupling member intermediate said upstream and downstream end portion, said magnifying lens forming a downstream boundary of said axially extending void region;
(c) a splash shield having a downstream terminal end carried by said coupling member, said splash shield having an axially extending chamber region therein, where said downstream end portion has a radially extending opening communicating with said chamber region and where said magnifying lens forms an upstream boundary of said chamber region, and where said magnifying lens has an orifice adjacent said peripheral edge to permit fluid communication between said axially extending void region and said axially extending chamber region.

2. The device recited in claim 1 where said coupling member is radially symmetrical to said axis of elongation.

3. The device recited in claim 1 where said radially extending opening is located adjacent said downstream terminal end of said splash shield.

4. The device recited in claim 1 where said splash shield has a multiplicity of drain ports located adjacent said downstream terminal end.

5. The device recited in claim 1 where said peripheral edge is so contoured and proportioned such that said orifice forms a part of said peripheral edge.

6. An improved system for both irrigating and examining a wound of the type having a manually compressible container, a dispensing outlet in the neck portion of said container, and a reservoir within said container for holding cleansing fluid, the improvement comprising:
(a) a coupling member having an axis of elongation, an upstream end portion, and a downstream end portion, where said upstream end portion is so dimensioned and proportioned to permit said upstream end portion to releasably couple with said dispensing outlet, said upstream end portion of said coupling member having a axially extending void region therein where said reservoir is in fluid communication with said axially extending void region upon coupling of said coupling member with said dispensing outlet;
(b) a magnifying lens having a peripheral edge carried by said coupling member intermediate said upstream and downstream end portion, said magnifying lens forming a downstream boundary of said axially extending void region;
(c) a splash shield having a downstream terminal end carried by said coupling member, said splash shield having an axially extending chamber region therein, where said downstream end portion has a radially extending opening communicating with said chamber region and where said magnifying lens forms an upstream boundary of said chamber region, and where said magnifying lens has an orifice adjacent said peripheral edge to permit fluid communication between said axially extending void region and said axially extending chamber region.

7. The improved system recited in claim 6 where said coupling member is radially symmetrical to said axis of elongation.

8. The improved system recited in claim 6 where said radially extending opening is located adjacent said downstream terminal end of said splash shield.

9. The improved system recited in claim 6 where said splash shield has a multiplicity of drain ports located adjacent said downstream terminal end.

10. The improved system recited in claim 6 where said peripheral edge is so contoured such that said orifice forms a part of said peripheral edge.

11. In combination:
a) A manually compressible container having a dispensing outlet for dispensing saline solution for cleansing wounds or lacerations from the reservoir of said container; and
b) a coupling member having an axis of elongation, an upstream end portion, and a downstream end portion where said upstream end portion is so dimensioned and proportioned to permit said upstream end portion to releasably couple with said dispensing outlet, said upstream end portion having an axially extending void region therein where said reservoir is in fluid communication with said axially extending void region upon coupling of said coupling member with said dispensing outlet, a magnifying lens have a peripheral edge carried by said coupling member intermediate said upstream and downstream end portions, said magnifying lens forming a downstream boundary of said axially extending void region, and a splash shield having a downstream terminal end carried by said coupling member, said splash shield having an axially extending chamber region therein, where said downstream end portion has a radially extending opening communicating with said chamber region and where said magnifying lens forms an upstream boundary of said chamber region, and where said magnifying lens has an orifice adjacent said peripheral edge to permit fluid communication between said axially extending void region and said axially extending chamber region.

12. The combination recited in claim 11 where said coupling member is radially symmetrical with said axis of elongation

13. The combination recited in claim 1 1 where said radially extending opening is located adjacent said downstream terminal end of said splash shield.

14. The combination recited in claim 11 where said splash shield has a multiplicity of drain ports located adjacent said downstream terminal end.

15. The combination recited in claim 11 where said peripheral edge is so contoured such that said orifice forms a part of said peripheral edge.

16. The device recited in claim 1 where said splash shield further comprises a multiplicity of radially and circumferentially spaced indicia integral with said splash shield and located adjacent said downstream terminal end and where the circumferential distance between each of said multiplicity of indicia is predetermined.

17. The improved system recited in claim 6 where said splash shield further comprises a multiplicity of radially and circumferentially spaced indicia integral with said splash shield and located adjacent said downstream terminal end and where the circumferential distance between each of said multiplicity of indicia is predetermined.

18. The combination recited in claim 11 where said splash shield further comprises a multiplicity of radially and circumferentially spaced indicia integral with said splash shield and located adjacent said downstream terminal end and where the circumferential distance between each of said multiplicity of indicia is predetermined.
